(19) United States
(12) Patent Application Publication
(10) Pub. No.: US 2020/0222240 A1
(43) Pub. Date: Jul. 16, 2020

(54) Illuminated Safety Glasses Assembly

(71) Applicant: Steven McManus, Livermore, CA (US)

(72) Inventor: Steven McManus, Livermore, CA (US)

(21) Appl. No.: 16/249,386

(22) Filed: Jan. 16, 2019

(51) Int. Cl. A61F 9/02; F21V 33/00; F21V 23/04; F21V 23/02

(52) U.S. Cl. CPC A61F 9/029; F21V 33/0064; A61F 9/026; F21V 23/02; F21V 23/04

(57) ABSTRACT

An illuminated safety glasses assembly for illuminating a work area when safety glasses are worn includes a pair of safety glasses that are worn on a user's face for protecting the user's eyes from fragments. A light emitter is coupled to the safety glasses to emit light outwardly therefrom for illuminating a work area. A switch is movably coupled to the safety glasses. The switch is electrically coupled to the light emitter for turning the light emitter on and off.

### CROSS-REFERENCE TO RELATED APPLICATIONS

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to safety glasses devices and more particularly pertains to a new safety glasses device for illuminating a work area when safety glasses are worn.

### BRIEF SUMMARY OF THE INVENTION

An embodiment of the disclosure meets the needs presented above by generally comprising a pair of safety glasses that are worn on a user's face for protecting the user's eyes from fragments. A light emitter is coupled to the safety glasses to emit light outwardly therefrom for illuminating a work area. A switch is movably coupled to the safety glasses. The switch is electrically coupled to the light emitter for turning the light emitter on and off.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of an illuminated safety glasses assembly according to an embodiment of the disclosure.

FIG. 2 is a front view of an embodiment of the disclosure.

FIG. 3 is a bottom view of an embodiment of the disclosure.

FIG. 4 is a left side exploded view of an embodiment of the disclosure.

FIG. 5 is a perspective view of an alternative embodiment of the disclosure.

### DETAILED DESCRIPTION OF THE INVENTION

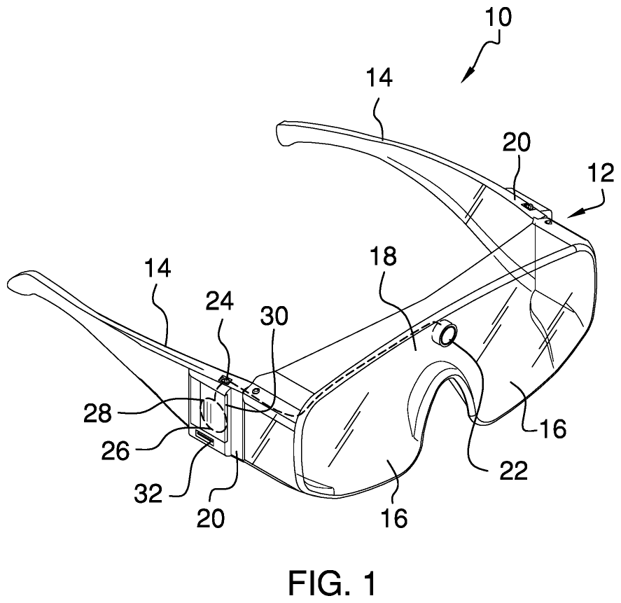

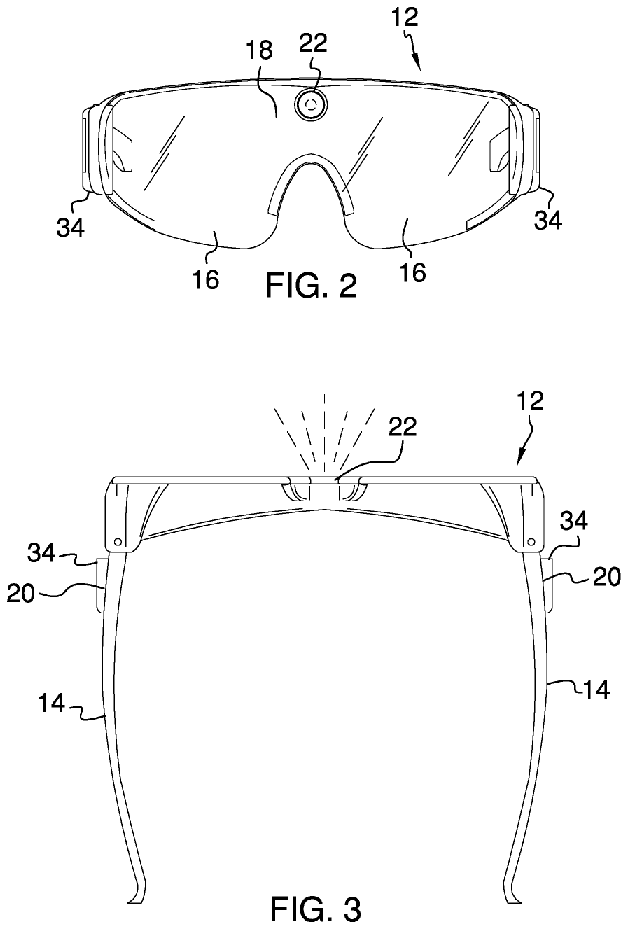

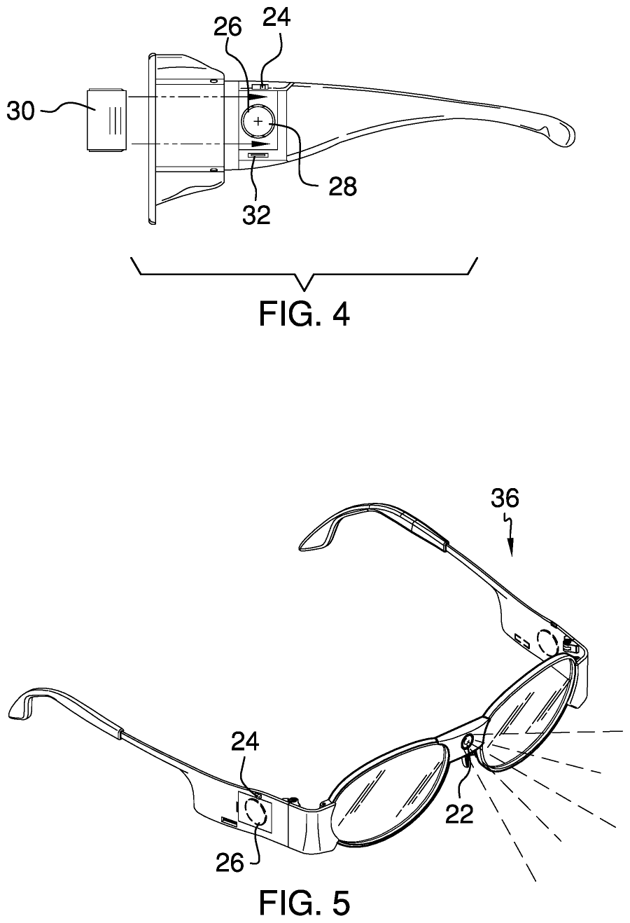

With reference now to the drawings, and in particular to FIGS. 1 through 5 thereof, a new safety glasses device embodying the principles and concepts of an embodiment of the disclosure and generally designated by the reference numeral 10 will be described.

As best illustrated in FIGS. 1 through 5, the illuminated safety glasses assembly 10 generally comprises a pair of safety glasses 12 that are worn on a user's face for protecting the user's eyes from fragments or projectiles. The safety glasses 12 includes a pair of arms 14 that is each hingedly coupled to a respective one of a pair of lenses 16 and a nose bridge 18 extending between the lenses 16. Each of the arms 14 has a temple section 20, and the safety glasses 12 may be safety glasses of any conventional design that conform to ANSI Z80.3/Z71.1. A light emitter 22 is coupled to the safety glasses 12 to emit light outwardly therefrom for illuminating a work area. The light emitter 22 is positioned on the nose bridge 18 and the light emitter 22 may comprise an LED or the like.

A switch 24 is movably coupled to the safety glasses 12 and the switch 24 is electrically coupled to the light emitter 22 for turning the light emitter 22 on and off. The switch 24 is positioned on the temple section 20 of a respective one of the arms 14. In this way the switch 24 does not interfere with the user's peripheral vision when the safety glasses 12 are worn. A power supply 26 is coupled to the safety glasses 12 and the power supply 26 is electrically coupled to the switch 24.

The power supply 26 comprises at least one battery 28, and the at least one battery 28 is positioned on the temple section 20 of a respective one of the arms 14. Thus, the at least one battery 28 does not interfere with the user's peripheral vision when the safety glasses 12 are worn. A battery cover 30 is removably coupled to the safety glasses 12 and the battery cover 30 is positioned on the temple section 20 of the arm 14 on which the at least one battery 28 is positioned. Moreover, the battery cover 30 covers the at least one battery 28 when the battery cover 30 is coupled to the safety glasses 12. The power supply 26 further includes a power port 32 that is recessed into the respective arm 14. The power port 32 is electrically coupled to the at least one battery 28, and a battery charger 34 can be plugged into the power port 32 for charging the at least one battery 28. The power port 32 may be a micro usb port or other similar port.

A pair of the power supplies 26 may be provided, and each of the power supplies 26 may be positioned on a respective one of the arms 14. Additionally, each of the power supplies 26 may include a battery housing 34 that is coupled to the respective arm 14. A pair of the switches 24 may be provided, and each of the switches 24 may be positioned on a respective one of the arms 14. In an alternative embodiment 36 as shown in FIG. 5, each of the light emitter 22, switch 24 and power supply 26 may be positioned on a pair of reading glasses 38 thereby enhancing the user's ability to read.

In use, the safety glasses 12 are worn to protect the user's eyes. The switch 24 is manipulated to turn on the light emitter 22 when the user is working in a darkened environment. Thus, the light emitter 22 illuminates the darkened environment to enhance the user's ability to work. Additionally, the switch 24 and the power supply 26 are positioned on the temple section 20 of the respective arm. Thus, neither of the switch 24 nor the power supply 26 impedes the user's peripheral vision when the safety glasses 12 are worn.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. An illuminated safety glasses assembly being configured to emit light toward a work area while protecting a user's eyes, said assembly comprising:
a pair of safety glasses being worn on a user's face for protecting the user's eyes from fragments, said safety glasses comprising
a pair of arms,
a pair of lenses, each of said arms being hingedly coupled to a respective one of said pair of lenses, and
a nose bridge, said nose bridge being integrally coupled to and extending between said lenses defining a unitary peripheral edge extending around said pair of lenses and said nose bridge;
a light emitter being coupled to said safety glasses wherein said light emitter is configured to emit light outwardly therefrom for illuminating a work area, said light emitter being positioned on said nose bridge within said unitary peripheral edge; and
a switch being movably coupled to said safety glasses, said switch being electrically coupled to said light emitter for turning said light emitter on and off.

2. The assembly according to claim 1, wherein each of said arms has a temple section.

3. The assembly according to claim 2, further comprising said switch is positioned on said temple section of a respective one of said arms wherein said switch is configured to not interfere with the user's peripheral vision when said safety glasses are worn.

4. (canceled)

5. The assembly according to claim 1, further comprising a power supply being coupled to said safety glasses, said power supply being electrically coupled to said switch, said power supply comprising at least one battery, said at least one battery being positioned on said temple section of a respective one of said arms wherein said at least one battery is configured to not interfere with the user's peripheral vision when said safety glasses are worn.

6. The assembly according to claim 5, further comprising a battery cover being removably coupled to said safety glasses, said battery cover being positioned on said temple section of said arm on which said at least one battery is positioned.

7. An illuminated safety glasses assembly being configured to emit light toward a work area while protecting a user's eyes, said assembly comprising:
a pair of safety glasses, said safety glasses comprising
a pair of arms, each of said arms having a temple section,
a pair of lenses, each of said arms being hingedly coupled to a respective one of said pair of lenses, and
a nose bridge, said nose bridge being integrally coupled to and extending between said lenses defining a unitary peripheral edge extending around said pair of lenses and said nose bridge;
a light emitter being coupled to said safety glasses wherein said light emitter is configured to emit light outwardly therefrom for illuminating a work area, said light emitter being positioned on said nose bridge within said unitary peripheral edge;
a switch being movably coupled to said safety glasses, said switch being electrically coupled to said light emitter for turning said light emitter on and off, said switch being positioned on said temple section of a respective one of said arms wherein said switch is configured to not interfere with the user's peripheral vision when said safety glasses are worn;
a power supply being coupled to said safety glasses, said power supply being electrically coupled to said switch, said power supply comprising at least one battery, said at least one battery being positioned on said temple section of a respective one of said arms wherein said at least one battery is configured to not interfere with the user's peripheral vision when said safety glasses are worn; and
a battery cover being removably coupled to said safety glasses, said battery cover being positioned on said temple section of said arm on which said at least one battery is positioned, said battery cover covering said at least one battery when said battery cover is coupled to said safety glasses.
